(19) United States
(12) Patent Application Publication
(10) Pub. No.: US 2012/0045511 A1
(43) Pub. Date: Feb. 23, 2012

(54) THERMOSENSITIVE HEPATITIS B VACCINE

(75) Inventors: Mi-Hua Tao, Taipei City (TW); Ho-Yuan Chou, Taipei City (TW); Hsin-Hsin Shen, Hsinchu County (TW); Tsai-Yu Lin, Changhua City (TW)

(73) Assignee: INDUSTRIAL TECHNOLOGY RESEARCH INSTITUTE, Hsinchu County (TW)

(21) Appl. No.: 12/858,305

(22) Filed: Aug. 17, 2010

(51) Int. Cl. A61K 9/00; A61K 39/29

(52) U.S. Cl. 424/484; 424/227.1

(57) ABSTRACT

A thermosensitive hepatitis B vaccine is provided. The thermosensitive hepatitis B vaccine includes an aqueous phase solution comprising a biodegradable thermosensitive hydrogel copolymer; a surface antigen of hepatitis B virus (HBsAg); and a bioactive substance. The thermosensitive hepatitis B vaccine of the disclosure is particularly suitable for being applied in the patients, which are low responsive or non-responsive to conventional hepatitis B vaccine, for enhancing the induction of cell-mediated immune responses and overcoming the HBsAg non-responsiveness.

### TECHNICAL FIELD

The disclosure relates to a hepatitis B vaccine, and more particularly to a thermosensitive hepatitis B vaccine.

### BACKGROUND

According to the World Health Organization (WHO), more than one-third of the world's population has been infected with hepatitis B virus (HBV) and over 350 million become chronic carriers, of whom 15-25% are at risk of developing HBV-associated liver diseases, including cirrhosis and hepatocellular carcinoma. Vaccination with the surface antigen of HBV (HBsAg) is the main strategy for effective control of the infection and viral transmission. The commercially available standard three-dose HBV vaccine, derived from plasma of HBV carriers or produced by recombinant DNA technology, results in a protective response of antibody to HBsAg (anti-HBs) in approximately 90% of healthy people. The success of HBsAg vaccine in protection from HBV infection is demonstrated by the pioneered national HBV immunization program to all newborns in Taiwan, which has brought down the rate of HBV chronic carriers from about 10% to less than 1%, and reduced the incidence of liver cancer in 12-14-old children by 75%. However, a small proportion (about 5-10%) of normal vaccine recipients and about 40-50% of patients on maintenance hemodialysis with depressed immune responses do not respond well to the current HBsAg vaccines. Mechanisms underlying non-responsiveness to HBsAg are not fully defined, but accumulating evidence from genetic studies indicate a close association between different HLA-DR alleles and specific low responsiveness in different ethnic populations.

Therefore, it is necessary to develop a novel hepatitis B vaccine for enhancing anti-HBsAg immune responses and overcoming the HBsAg non-responsiveness.

### SUMMARY

The disclosure provides a thermosensitive hepatitis B vaccine, except to the surface antigen of hepatitis B virus (HBsAg) and the bioactive substance (serving as a adjuvant), includes an aqueous phase solution comprising a biodegradable thermosensitive hydrogel copolymer. Due to the thermosensitivity, the novel hepatitis B vaccine can be a flowable pharmaceutical form and be directly injected into the patient. After injecting, the hepatitis B vaccine can be transformed into a hydrogel due to the increased viscocity resulting from body temperature, and can generally release the carried drugs to provide effectivity in immune responses. Further, since the thermosensitive hepatitis B vaccine of the disclosure includes an aqueous phase solution comprising a biodegradable thermosensitive hydrogel copolymer, the anti-Hepatitis B virus immune responses can be enhanced. Therefore, the thermosensitive hepatitis B vaccine of the disclosure is particularly suitable for being applied in the patients, which are low responsive or non-responsive to conventional hepatitis B vaccine, for enhancing the induction of cell-mediated immune responses and overcoming the HBsAg non-responsiveness.

The hepatitis B vaccine of the disclosure includes an aqueous phase solution comprising a biodegradable thermosensitive hydrogel copolymer, hepatitis B virus (HBsAg), and at least one bioactive substance. Particularly, the biodegradable thermosensitive hydrogel copolymer can be a di-block or triblock co polymer prepared by polymerizing polyethylene glycol (PEG), lactide (LA), and glycolide (GA). Further, the biodegradable thermosensitive hydrogel copolymer can be PEG-PLGA, PEG-PLGA-PEG, PLGA-PEG-PLGA, or combinations thereof. The polyethylene glycol can be polyethylene glycol polymer or methoxy-poly(ethylene glycol), and can have a molecular weight of 350-2000 g/mole. Further, poly lactide-co-glycolide can be polymers or copolymers derived from D,L-Lactide, D-Lactide, L-Lactide, D,L-Lactic acid, D-Lactic acid, L-Lactic acid, glycolide, β-propiolactone, δ-valerolactone, or ε-caprolactone. PLGA can have a molecular weight of 1000-2500 g/mole.

Moreover, the biodegradable thermosensitive hydrogel copolymer is uniformly distributed in water to form an aqueous phase solution, wherein the concentration of the hydrogel copolymer can be of between 0.05-0.5g/ml. In embodiment of the disclosure, the bioactive substance can be granulocyte-macrophage colony-stimulating factor (GM-CSF or GM). The concentration of the surface antigen of hepatitis B virus (HBsAg) among the vaccine can be of between 0.1-50 μg/ml, and the concentration of the granulocyte-macrophage colony-stimulating factor among the vaccine can be of between 1×10⁴-1×10⁶U.

The thermosensitive hepatitis B vaccine of the disclosure exhibits reverse thermal gelation properties and has a lower critical solution temperature (LCST) of between 10-90° C., preferably 20-45° C. The thermosensitive hepatitis B vaccine behaves as a liquid with low viscocity below the critical solution temperature. After heating, the viscocity of the biodegradable copolymer hydrogen quickly rises, undergoing a reversible liquid-gel (or semi-solid) phase transition. It should be noted that, after long-period degradation, the biodegradable thermosensitive hydrogel copolymer of the hepatitis B vaccine is non-toxicity since the hydrolysate has a pH value of more than 5.0.

A detailed description is given in the following embodiments with reference to the accompanying drawings.

### BRIEF DESCRIPTION OF THE DRAWINGS

The disclosure can be more fully understood by reading the subsequent detailed description and examples with references made to the accompanying drawings, wherein:

FIG. 1 is a graph plotting the anti-HBsAg titer of mice respectively immunized with various HBsAg treatments.

FIG. 2 is a graph plotting the anti-HBsAg titer of mice respectively immunized with the hepatitis B vaccine of the disclosure and the conventional hepatitis B vaccine (H-B-Vax II).

FIG. 3 is a graph plotting the anti-HBsAg titer of various mice.

FIG. 4 is a graph plotting the anti-HBsAg titer of B10.M (H-2f) mice respectively immunized with various HBsAg treatments at first and second injections.

FIG. 5 is a graph plotting the stimulation index of mice respectively immunized with various HBsAg treatments.

### DETAILED DESCRIPTION

The following examples are intended to illustrate the disclosure more fully without limiting the scope of the disclosure, since numerous modifications and variations will be apparent to those skilled in this art.

Preparation of Aqueous Phase Solution Comprising a Biodegradable Thermosensitive Hydrogel Copolymer

### EXAMPLE 1

A glass reactor (250 ml volume) connecting with a condenser, a heater, and a thermostat was provided, wherein educts of the condenser wrapped with heating tape looped back and rejoined to the reactor. 26 g of methoxy-poly(ethylene glycol) (with a molecular weight of 550 g/mole), 50 g of lactide and 17 g of glycolide were added in the reactor, and the temperature was elevated slowly for complete dissolution. When the temperature reached and was sustained at 160° C., 37 μl of catalyst (stannous 2-ethyl-hexanoate) was added. After polymerization was performed for 8 hr, the product is precipitated with diethyl ether/n-hexane (v/v=1/1) to form a translucent colloid. The residual monomers are washed for three times and dried in a vacuum for 24 hr at 40° C., obtaining a di-block copolymer mPEG₅₅₀-PLGA₁₄₀₅ (biodegradable thermosensitive hydrogel copolymer).

Next, 15 g of di-block copolymer mPEG₅₅₀-PLGA₁₄₀₅ was added into 85 ml of water, preparing an aqueous phase solution comprising a biodegradable thermosensitive hydrogel copolymer (the biodegradable thermosensitive hydrogel copolymer has a weight percentage of 15 wt %).

Preparation of Thermosensitive Hepatitis B Vaccine

### EXAMPLE 2

2 μg of HBsAg (yeast-derived recombinant), and 5.4 μg (or 4.5×10⁴U) of mouse GM-CSF (produced in Pichia pastoris yeast) were mixed with 0.2 ml aqueous phase solution (comprising the biodegradable thermosensitive hydrogel copolymer mPEG₅₅₀-PLGA₁₄₀₅) prepared by Example 1, preparing a thermosensitive hepatitis B vaccine.

Effect on Hepatitis B Virus

Mice were divided into groups (n=6) receiving one of the following vaccines: (1)2 μg HBsAg in saline, (2)2 μgHBsAg+5.4 μg GM-CSF in saline, (3)2 μg HBsAg in hydrogel copolymer (prepared in Example 1), (4) hepatitis B vaccine (2 μg HBsAg+5.4 μg GM-CSF in hydrogel copolymer, (5) Gel/HBsAg and Gel/GM injected at two separate sites; and (6)2 μg of the commercial yeast-derived recombinant HBsAg, H-B-Vax II (Merck Sharp & Dohme, West Point, Pa.), which was formulated with aluminum hydroxide ·All BALB/c mice were immunized at 6 to 8 weeks of age.

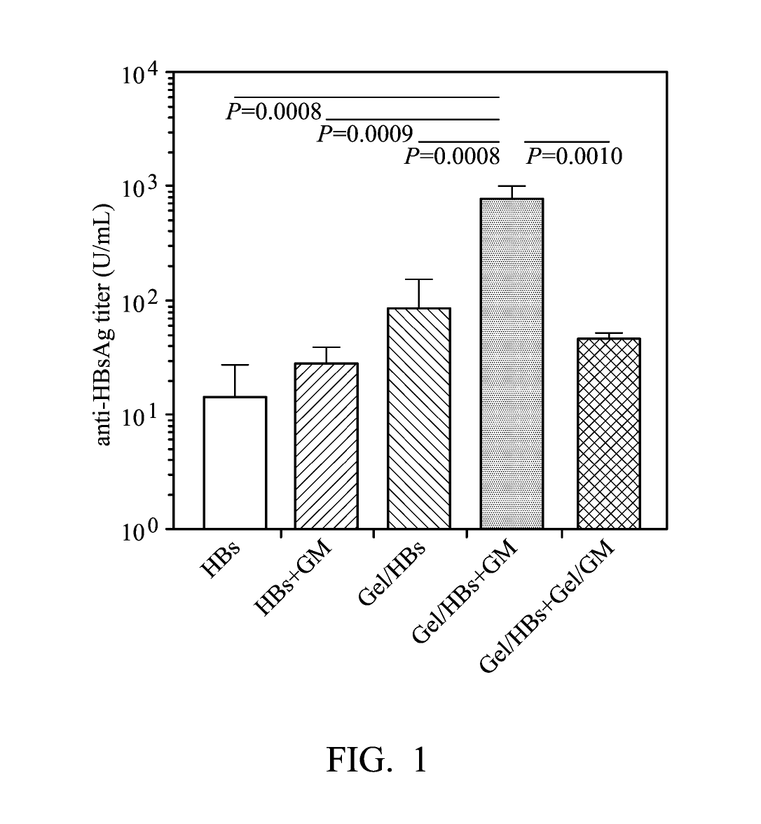

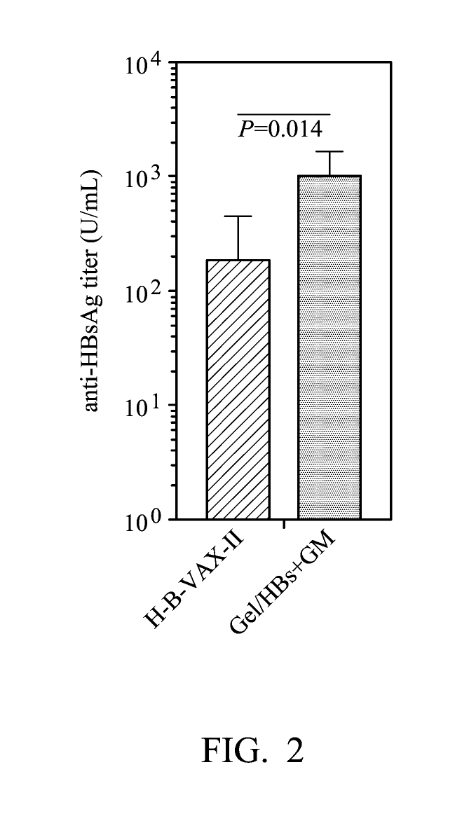

Next, sera were collected from each group to measure HBs-specific antibodies. As shown in FIG. 1, mice immunized with HBsAg alone produced only low titers of anti-HBsAg Ab (14±13 U/ml, mean±SD). Codelivery of GM-CSF with HBsAg (HBsAg+GM group) increased anti-HBsAg titers by 2-fold (28±10 U/ml), while hydrogel-delivered HBsAg (Gel/HBsAg group) produced 6-fold more anti-HBsAg antibodies (84±69 U/ml) compared to that in the HBsAg group. The most significant result was obtained by the vaccine provided by Example 2 (Gel/HBsAg+GM group), which profoundly increased anti-HBsAg titer to 773±227 U/ml, being 56-, 27-, and 9-fold higher than those obtained in the HBsAg (p=0.0008), HBsAg+GM (p=0.0009), and Gel/HBsAg (p=0.0008) groups. The adjuvant activity of GM-CSF was lost when Gel/HBsAg and Gel/GM were injected at two separate sites, which produced a far less anti-HBsAg titer (45±6 U/ml) compared with the Gel/HBsAg+GM group (p=0.001, FIG. 1A), highlighting the importance of local GM-CSF activity in promoting immune responses. The the vaccine of the disclosure (Gel/HBsAg+GM) was also compared with a commercially recombinant HBsAg vaccine, H-B-VAXII, which was formulated with aluminum hydroxide as adjuvant. As shown in FIG. 2, mice immunized with Gel/HBsAg+GM vaccine produced much higher anti-HBsAg titers (1040±660 U/ml) compared with that achieved by the H-B-VAXII (182±63 U/ml, p=0.014) vaccine.

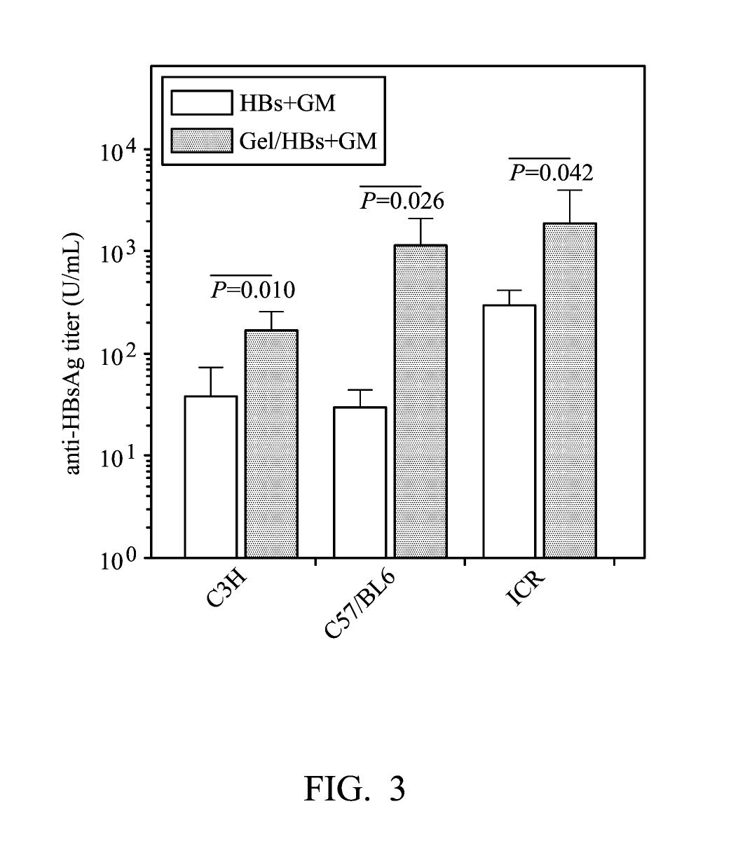

To confirm that the antibody enhancement effect was not restrict to BALB/c mice (haplotype H-2d), several inbred [C57BL/6 (haplotype H-2b), C3H/HeN (haplotype H-2k)] and outbred (ICR) mice were immunized with the same dose of HBsAg and GM-CSF delivered with or without hydrogel. As shown in FIG. 3, the vaccine of the disclosure (Gel/HBsAg+GM) significantly improved anti-HBsAg titers in mice of different genetic background, with 4-fold increase in C3H/HeN (p=0.010), 34-fold increase in C57BL/6 (p=0.026), and 6-fold increase in ICR (p=0.042), compared with those immunized with a simple mixture of HBsAg+GM.

Next, in order to conform whether T cell immune responses can be enhanced by the vaccine of the disclosure, groups of BALB/c mice were injected twice at 2-week with 2 μg of HBsAg in different vaccine formulations as described above. One week after the second immunization, splenocytes were examined for proliferation in response to specific (HBsAg) and non-specific (bovine serum albumin, BSA) antigen stimulation. The results are shown in Table 1. Splenic lymphocytes derived from the HBsAg group demonstrated dose-dependent proliferative responses to an increasing HBsAg, with an average peak stimulation index of about 7.0 when HBsAg was at 10 μg/ml. Compared with the HBsAg group, immunization with HBsAg+GM or Gel/HBsAg produced slightly higher cellular proliferation, with the average peak stimulation index (HBsAg at 10 μg/ml) increased to 9.1 and 11.0, respectively. Not surprisingly, the most significant enhancement of T cell proliferation was achieved in animals immunized with Gel/HBsAg+GM, which had an average peak stimulation index (HBsAg at 10 μg/ml) of 43.3. The T cell responses were specific to HBsAg, because all mice of the different groups failed to respond to the control protein BSA at a much higher concentration (30 μg/ml).

TABLE 1
T-cell stimulation index with stimulant

| | HBsAg | | | BSA |
|---|---|---|---|---|
| Antigen | 10 μg/ml | 3 μg/ml | 1 μg/ml | (30 μg/ml) |
| HBsAg | 7.0 ± 0 | 2.5 ± 0.6 | 1.4 ± 0.4 | 1.0 ± 0.1 |
| HBsAg + GM | 9.1 ± 2.4 | 6.5 ± 1.9 | 4.0 ± 3.2 | 1.3 ± 1.1 |
| Gel/HBsAg | 11.0 ± 1.0 | 6.1 ± 3.8 | 2.3 ± 0.1 | 1.6 ± 0.6 |
| Gel/HbsAg + GM | 43.3 ± 2.1 | 31.0 ± 3.5 | 17.8 ± 4.4 | 1.1 ± 0.1 |

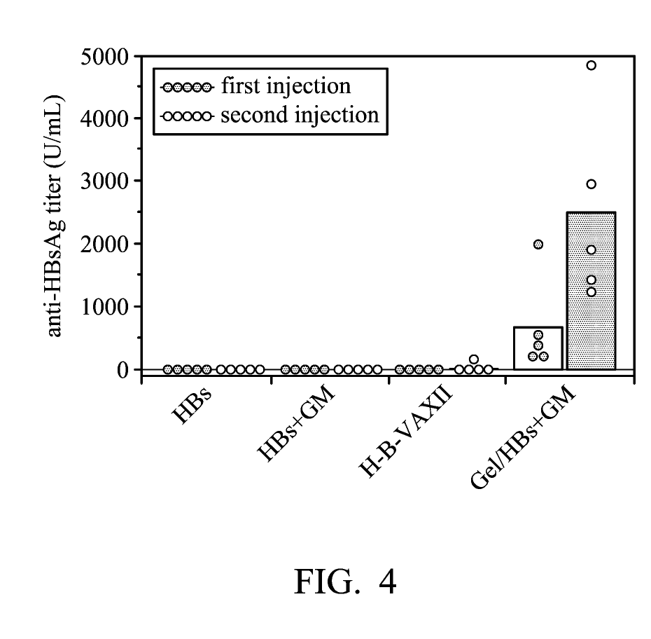

Next, in order to conform whether the MHC-restricted non-responsiveness to HbsAg can be overcome by the vaccine of the disclosure, groups of B10.M (H-2f) mice were given two doses of HBsAg in different formulations as described above. Sera were collected at 4 weeks after the first injection and 2 weeks after the second injection, and assayed for the presence of anti-HB Abs by ELISA. As shown in FIG. 4, the vaccine of the disclosure (Gel/HBsAg+GM) was able to elicit significant anti-HBs Abs in all mice after the first immunization with an average titer of 659±529 U/mL, and the titer was further increased about 4-fold to an average of 2471±1136 U/mL after the booster immunization. In contrast, all the other HBsAg vaccines, including HBs alone, HBsAg+GM, and the commercial H-B-VAXII, failed to induce detectable anti-HBs titers in B10.M mice, except one animal in the H-B-VAXII group, which produced a weak anti-HBs titer after booster immunization, referring to FIG. 4.

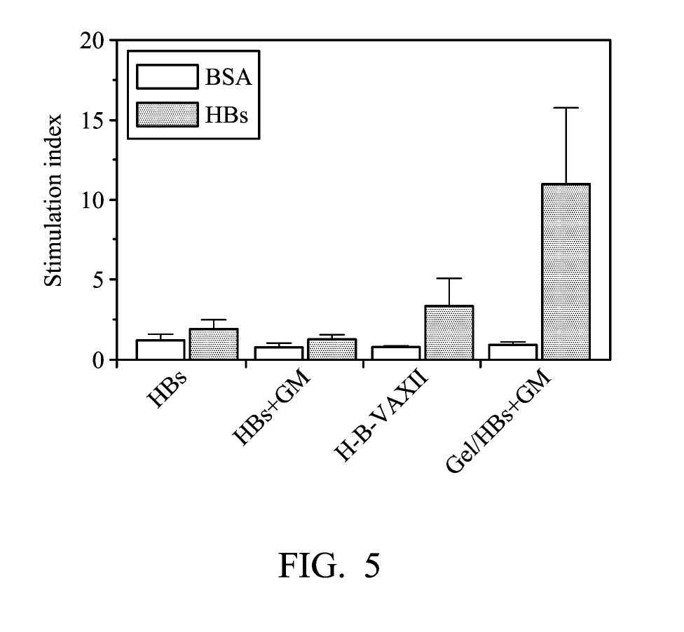

Induction of cell-mediated immune responses in B10.M mice was then investigated to prove the immune ability of the vaccine of the disclosure (Gel/HBsAg+GM). The mice were immunized twice as above and analyzed two weeks after the second immunization. As shown in FIG. 5, B10.M mice immunized with Gel/HBsAg+GM vaccine developed a significant T cell proliferative response to HBsAg (an average stimulation index of 11.0±4.9), but not the control BSA protein (an average stimulation index of 1.0±0.2). Immunization of H-B-VAXII produced a low but significant HBs-specific T cell response (an average stimulation index of 3.3±1.8), while HBs alone or HBsAg+GM did not induce detectable T cell proliferative responses to HBs. These results demonstrate that the vaccine of the disclosure (Gel/HBsAg+GM) is effective in breaking the MHC-restricted non-responsiveness to HBsAg in both the humoral and cellular arms of immunity.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A thermosensitive hepatitis B vaccine, comprising:
an aqueous phase solution comprising a biodegradable thermosensitive hydrogel copolymer;
a surface antigen of hepatitis B virus (HBsAg); and
a bioactive substance.

2. The thermosensitive hepatitis B vaccine as claimed in claim 1, wherein the biodegradable thermosensitive hydrogel copolymer is a di-block or tri-block copolymer preparing by polymerizing polyethylene glycol, lactide, and glycolide.

3. The thermosensitive hepatitis B vaccine as claimed in claim 2, wherein the biodegradable thermosensitive hydrogel copolymer comprises PEG-PLGA, PEG-PLGA-PEG, PLGA-PEG-PLGA, or combinations thereof.

4. The thermosensitive hepatitis B vaccine as claimed in claim 3, wherein the biodegradable thermosensitive hydrogel copolymer comprises PEG moieties having a molecular weight of 350-2000 g/mole.

5. The thermosensitive hepatitis B vaccine as claimed in claim 3, wherein the biodegradable thermosensitive hydrogel copolymer comprises PLGA moieties having a molecular weight of 1000-2500 g/mole.

6. The thermosensitive hepatitis B vaccine as claimed in claim 1, wherein the concentration of the hydrogel copolymer is of between 0.05-0.5 g/ml, based on the volume of the the aqueous phase solution.

7. The thermosensitive hepatitis B vaccine as claimed in claim 1, wherein the bioactive substance is granulocyte-macrophage colony-stimulating factor (GM-CSF).

8. The thermosensitive hepatitis B vaccine as claimed in claim 1, wherein the concentration of the surface antigen of hepatitis B virus (HBsAg) among the vaccine is of between 0.1-50 μg/ml.

9. The thermosensitive hepatitis B vaccine as claimed in claim 7, wherein the concentration of the granulocyte-macrophage colony-stimulating factor among the vaccine can be of between 1×10⁴-1×10⁶U.
